(19) United States
(12) Patent Application Publication
(10) Pub. No.: US 2020/0200697 A1
(43) Pub. Date: Jun. 25, 2020

(54) ELECTROCHEMICAL BIOSENSORS

(71) Applicant: SUN CHEMICAL CORPORATION, Parsippany, NJ (US)

(72) Inventors: Nabil El Murr, Nantes (FR); Carmen Creanga, Orée d'Anjou (FR); Robin Pittson, St. Briavels (GB)

(73) Assignee: SUN CHEMICAL CORPORATION, Parsippany, NJ (US)

(21) Appl. No.: 16/613,151

(22) PCT Filed: Jun. 5, 2018
(86) PCT No.: PCT/US18/36048

§ 371 Date: Nov. 13, 2019

Related U.S. Application Data
(60) Provisional application No. 62/516,194, filed on Jun. 7, 2017.

(51) Int. Cl. G01N 27/327; C12Q 1/00

(52) U.S. Cl. CPC G01N 27/3272; C12Q 1/006; G01N 27/3273

(57) ABSTRACT

The present invention provides electrochemical biosensors for measuring low concentrations of analytes in biological fluid samples. The biosensors comprise one or more working electrodes, a reference electrode, a counter electrode, and a bio-cocktail. The bio-cocktail comprises one or more enzymes and one or more redox mediators in a buffered solution. The present invention also provides methods of measuring low concentrations of analytes in biological fluid samples using the electrochemical biosensors of the invention.

### CROSS-REFERENCE TO RELATED APPLICATIONS

The present application is a § 371 National Phase application based on PCT/US2018/036048 filed Jun. 5, 2018, which claims the benefit of U.S. Provisional Application No. 62/516,194, filed Jun. 7, 2017 the subject matter of each of which is incorporated by reference in their entirety.

### FIELD OF THE INVENTION

The present invention is related to an electrochemical biosensor for measurement of the concentration of analytes, such as glucose and sucrose, in biological fluids. The present invention is suitable to measure low levels of analytes, for example as are found in biological fluids such as saliva, sweat, and tears. Using the electrochemical sensors of the present invention, the concentrations of analytes can advantageously be measured in cathodic mode, and at potentials that avoid interference from other oxidizable species. The biosensors and methods of the present invention are also suitable for measuring low concentrations of analytes, such as glucose and sucrose, in various food and agricultural products.

### BACKGROUND OF THE INVENTION

Test strips are currently being used extensively in the art of testing glucose in blood samples. One common application is for diabetics who need to frequently test and monitor their levels so as to adjust and maintain healthy levels. The current practices involve the need to use a needle to constantly prick the skin and release a small amount of blood for testing purposes. This is unsatisfactory for many reasons.

In a more recent development, a glucose monitor can be embedded inside the body to provide continuous blood testing and feedback to the user. Although this approach is favored by some, it is an invasive technique and carries its own share of inherent problems.

It would be advantageous if there was a test in which body fluids (e.g. saliva, tears, sweat) that contain glucose levels considerably lower than blood could be used as the test sample. However, current methods do not allow for easy detection of the much lower levels of glucose when compared to blood. Typical saliva and tears glucose levels are in the magnitude of about 1/100^{th} of the levels in blood.

Several attempts have been made to develop test methods and devices for detection of analytes in blood, and low levels of analytes, e.g. glucose, in bodily fluids such as saliva. They include a range of devices.

U.S. Pat. No. 5,770,028 describes a solid-state, multi-use electrochemical sensor generally incorporating enzyme electrodes having a metallized carbon base and an overlying silicone-containing protective glucose and/or lactate permeable membrane. They describe formulating cellulose acetate into a paste for use in an electrode. No redox mediators are used.

U.S. Pat. No. 6,623,698 discloses a biosensor electrical toothbrush having a head with a test channel and a renewable biosensor system within the test channel. The device can be used for performing routine saliva tests. No redox mediators are used.

US 2001/0023324 describes a device for stimulating saliva, collecting the saliva, and measuring the concentration of glucose. The preferred embodiment is in the form of a test strip. The device contains an absorbent matrix in contact with a threshold-type indicator film. The film contains glucose oxidase and peroxidase, and reagents that change color when exposed to glucose. No redox mediators are used.

US 2008/0177166 discloses an amperometric glucose sensor strip system. The sensor strip utilizes a platinum film for the detection of hydrogen peroxide generated from the breakdown of glucose by glucose oxidase in a biological sample. It is a single enzyme system, and no redox mediators are used.

US 2014/0197042 describes an electrochemical sensor for determining glucose concentration in a liquid sample. The sensor is optionally coated in the sample placement area with a plurality of sensor elements. The sensor elements include or consist of single-walled carbon nanotubes, graphite, graphene, carbon nanofibers, carbon nanowires and combinations thereof. The sensor elements, or the working electrode if no sensor elements are present, are functionalized with a coating containing glucose oxidase. It is a single enzyme system, and no redox mediators are used.

U.S. Pat. No. 6,767,441 discloses an electrochemical sensor, for measuring the concentration of various analytes in biological fluids, that combines at least one enzyme with a peroxidase and a redox mediator. Ferrocyanides are preferred as the redox mediator. The reference electrode must be loaded with a redox mediator for the working electrode(s) to work properly. The electrochemical sensor can be operated at a lower voltage so that interfering species are not oxidized. The electrochemical sensor is composed of laminated layers, with conductive conduits, and cutouts to allow contact of the sample with the electrodes. The working electrodes and the reference electrode are each in contact with separate conductive conduits.

Another use for glucose and sucrose testing involves the testing of foods and/or vegetables. One particular application is for potatoes, where traditional glucose testing devices are not geared to accurately test the relatively low levels of glucose and sucrose present in potatoes, compared to the higher levels in blood. It has been increasingly important to measure the levels of glucose and sucrose in food, especially potatoes, as glucose and sucrose can combine with aminoacid (asparagine) to produce acrylamide materials, which have been identified as carcinogens (see Official Journal of the European Union L304/24 21 November 2017). Therefore, in order to minimize the production of acrylamide, it is important to have a means for easy and accurate detection of the levels of glucose and sucrose. This could also be an important tool to meet government thresholds for these materials (Creanga and El Murr. Disposable biosensor for measuring sugars in potatoes and assessing acrylamide formation. Sensor Letters, Vol. 9, 1-4 (2011)). Creanga and El Murr describe an electrochemical system utilizing test strips. The test strips are functionalized with a dual enzyme system, glucose oxidase and horseradish peroxidase, and utilizing a redox mediator to overcome interference from other oxidizing species.

What is still needed is a technology that can be used for easily detecting lower concentrations of sugars (e.g. glucose and sucrose) in other body fluids, such as saliva, tears, and sweat. This would provide an easy, painless and non-invasive approach for measuring and monitoring sugar levels in humans and other animals.

### SUMMARY OF THE INVENTION

The present invention provides electrochemical biosensors for measuring low concentrations of analytes in biological fluid samples. The present invention also provides methods for measuring low concentrations of analytes in biological fluid samples using the electrochemical biosensors of the present invention.

In a particular aspect, the present invention provides a method of measuring the concentration of one or more analytes in biological fluids, comprising:
a) placing a biological fluid sample on an electrochemical biosensor, wherein the electrochemical biosensor comprises:
   i. a support member substrate;
   ii. an electrochemical cell disposed on the support member substrate (transducer) comprising one or more working electrodes, a counter electrode, and a reference electrode;
   iii. a bio-cocktail disposed on the electrochemical cell, wherein the bio-cocktail comprises:
      a. one or more enzymatic catalysts selected from the group consisting of one or more oxidases (OX), a mutarotase (MUT), an invertase (INV), and one or more peroxidases (POX); and
      b. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂;
b) applying a voltage to the electrochemical biosensor; and
c) measuring the current output;
wherein the current output is a function of the concentration of the analyte in the biological fluid.

In another aspect, the present invention provides an electrochemical biosensor for measuring concentration of glucose in biological fluid samples, comprising:
a) a support member substrate;
b) an electrochemical cell disposed on the support member substrate (transducer) comprising one or more working electrodes, a counter electrode, and a reference electrode;
c) a bio-cocktail disposed on the electrochemical cell, wherein the bio-cocktail comprises:
   i. glucose oxidase;
   ii. mutarotase;
   iii. peroxidase; and
   iv. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂.

In yet another aspect, the present invention provides an electrochemical biosensor for measuring concentration of sucrose in biological fluid samples, comprising:
a) a support member substrate;
b) an electrochemical cell disposed on the support member substrate (transducer) comprising one or more working electrodes, a counter electrode, and a reference electrode;
c) a bio-cocktail disposed on the electrochemical cell, wherein the bio-cocktail comprises:
   i. glucose oxidase;
   ii. mutarotase;
   iii. invertase;
   iv. peroxidase; and
   v. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂.

In one embodiment, the bio-cocktail is disposed only on one or more of the working electrodes.

In a certain embodiment, the voltage is applied to one or more of the working electrodes.

In certain embodiments, the analyte is glucose, sucrose, or both glucose and sucrose.

### BRIEF DESCRIPTION OF THE DRAWINGS

FIG. 1 shows the reaction by which a current is generated to measure concentration of an analyte in a biological fluid sample in a first generation electrochemical biosensor.

FIG. 2 shows the reaction by which a current is generated to measure concentration of an analyte in a biological fluid sample in a second generation electrochemical biosensor.

FIG. 3 shows the redox reaction for generating current to measure the concentration of analytes in biological fluid samples, as well as competing redox reactions.

FIG. 4 shows the redox reaction between a ferrocene redox mediator and glucose in the presence of glucose oxidase.

FIG. 5 shows the current generated by redox reaction of various ferrocene redox mediator derivatives with glucose in the presence of glucose oxidase.

FIG. 6 shows the cyclic voltammetric current produced by various ferrocene redox mediator derivatives.

FIG. 7 shows the cyclic voltammetric current produced by redox reaction of various ferrocene redox mediator derivatives with glucose in the presence of glucose oxidase.

FIG. 8 shows the electrochemical mechanism EC′ of the redox reaction between ferrocene derivatives with glucose in the presence of glucose oxidase.

FIG. 9 shows the chronoamperometry curves recorded, at +0.3 V vs. Ag/AgCl, of various ferrocene redox mediator derivatives alone, then in the presence of glucose and glucose oxidase.

FIG. 10 shows the glucose calibration curves using biosensors operating with different ferrocene redox mediator derivatives.

FIG. 11 shows the reactions taking place on the biosensors when the redox mediator used is Fc-(CH₂—CO₂H)₂.

FIG. 12 shows a full card of screen printed electrochemical biosensor transducers.

FIG. 13 shows printing of three electrodes on an electrochemical biosensor substrate.

FIG. 14 shows printing of two electrodes on an electrochemical biosensor substrate.

FIG. 15 shows a chronoamperometric graph of current versus time, for a test in saliva.

### DETAILED DESCRIPTION OF THE INVENTION

It is to be understood that the foregoing general description, and the following detailed description are exemplary and explanatory only, and are not restrictive of any subject matter claimed.

Unless otherwise defined, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety for any purpose.

The present invention provides a method of measuring the concentration of analytes, such as glucose and sucrose, in biological fluids. The present invention provides an electrochemical biosensor test strip, which comprises an electrochemical cell, wherein the electrochemical cell (transducer) comprises one or more working electrodes, a reference electrode, and a counter electrode. A bio-cocktail containing one or more oxidases, one more peroxidases, other enzymes, and a ferrocene redox mediator is provided. The test strips are particularly useful for detecting the low concentrations of glucose, for example, present in biological fluids such as saliva, sweat, and tears.

### Definitions

In this application, the use of the singular includes the plural unless specifically stated otherwise. As used herein, the singular forms “a,” “an” and “the” are intended to include plural forms as well, unless the context clearly indicates otherwise.

In this application, the use of “or” means “and/or” unless stated otherwise.

As used herein, the terms “comprises” and/or “comprising” specify the presence of the stated features, integers, steps, operations, elements, and/or components, but do not preclude the presence or addition of one or more other features, integers, steps, operations, elements, components, and/or groups thereof. Furthermore, to the extent that the terms “includes,” “having,” “has,” “with,” “composed,” “comprised” or variants thereof are used in either the detailed description or the claims, such terms are intended to be inclusive in a manner similar to the term “comprising.”

As used herein, ranges and amounts can be expressed as “about” a particular value or range. “About” is intended to also include the exact amount. Hence “about 5 percent” means “about 5 percent” and also “5 percent.” “About means within typical experimental error for the application or purpose intended.

Throughout this disclosure, all parts and percentages are by weight (wt % or mass % based on the total weight) and all temperatures are in ° C. unless otherwise indicated.

As used herein, the terms “biosensor,” “glucose sensor,” and the like may be used interchangeably, and refer to the biosensor of the invention, whether to measure glucose, sucrose, fructose, or other analytes in biological fluids.

As used herein, the terms “anodic mode” and “oxidation mode” refer to a positive current measured when the potential (either at positive or negative potential vs the reference) is applied to the working electrode compared to the reference electrode. In anodic or oxidation mode, electrons are transferred from the reagent (e.g. redox mediator) to the working electrode.

As used herein, the terms “cathodic mode” and “reduction mode” refer to a negative current measured when the potential (either at positive or negative potential vs the reference) is applied to the working electrode compared to the reference electrode. In cathodic or reduction mode, electrons are transferred from the working electrode to the reagent (e.g. redox mediator).

### Electrochemical Biosensor and Use Thereof

Presently, the majority of glucose electrochemical biosensors are based on anodic detection in chronoamperometric mode and their selectivity is very much dependent on the interfering species which are detectable at the potential applied to the working electrode. Interfering species include oxygen, acetaminophen, ascorbic acid, and the like. Furthermore, many oxidized redox mediators may interfere with the electrochemical detection in and of themselves.

An electrochemical biosensor typically comprises three electrodes: a working electrode, a reference electrode, and a counter electrode. The redox reaction that is being monitored occurs at the surface of the working electrode. The surface of the working electrode contains the biorecognition element (e.g. the bio-cocktail of the present invention). The reference electrode has a constant and well-known potential. The reference electrode is often an Ag/AgCl electrode. The “applied potential” in an electrochemical biosensor method refers to the difference between the potential applied to the working electrode, such as for example by a potentiostat, compared to the standard potential of the reference electrode. Thus, when we speak of a positive (anodic or oxidative) potential, it means that the potential applied to the working electrode is higher than the potential at the reference electrode (i.e. the difference is positive number greater than 0 V). When we speak of a negative (cathodic or reductive) potential, it means that the potential applied to the working electrode is less than the standard potential at the reference electrode (i.e. the difference is a negative number, less than 0 V). The counter electrode is a current sink. The counter electrode prevents a current threshold by the reference electrode. In some instances, the reference electrode and the counter electrode may be the same electrode.

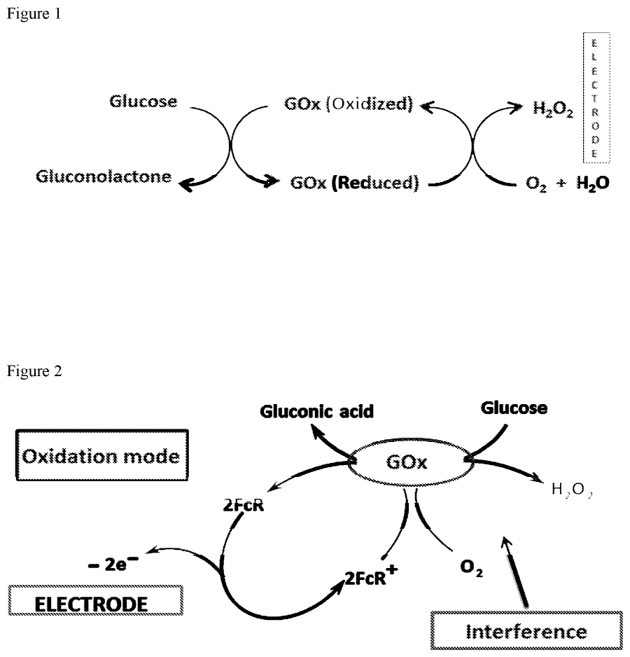

One of the first generation of glucose electrochemical biosensors used glucose oxidase (GOx) enzyme to transform glucose in the presence of oxygen into gluconolactone and to generate hydrogen peroxide (H₂O₂) which is detected and quantified on the surface of the working electrode of the biosensor. An applied potential, at the working electrode, of roughly +0.6 V vs. an Ag/AgCl reference electrode was necessary to quantify the amount of H₂O₂ produced. The amount of H₂O₂ measured is equal to the amount of glucose present in the sample (see FIG. 1). However, most biochemical compounds (e.g. enzymes) do not readily exchange electrons with the working electrode. A second generation of electrochemical biosensors, using redox mediators, was developed, as described below.

A second generation used redox mediators as a relay between GOx and the electrode surface permitting a decrease of the working potential and avoiding some of the interferences of other products present in the glucose samples. The redox mediator, which can exist in stable reduced and oxidized forms (eg. ferrocene derivatives), can assist in transferring electrons between the electrode and a redox enzyme. Ferrocene derivatives (FcR) are probably the most popular mediators used in electrochemical glucose biosensors (see FIG. 2). Most biochemical compounds (e.g. enzymes) do not readily exchange electrons with the working electrode. The redox mediator, which can exist in reduced or oxidized stable forms, can assist in exchanging electrons between the electrode and a redox enzyme. In this single enzyme system, the oxidized form of the redox mediator, FcR⁺ is reacted with the glucose in the presence of GOx to produce the reduced form, FcR (see FIG. 4). When the FcR comes in contact with the working electrode having an anodic potential (i.e. oxidation mode), the FcR loses electrons to the electrode (i.e. transfers electrons), and is transformed back to the oxidized, FcR⁺ form. The current generated by this transfer of electrons from FcR to the electrode is proportional to the concentration of glucose in the sample. However, the simultaneous presence of oxygen, glucose and GOx affects the correct measurement of glucose by generating gluconic acid, thus reducing the concentration of glucose. The interference of this reaction consumes as much glucose as that of the oxygen present in solution, and thus makes it difficult to measure the low concentrations of glucose. The H₂O₂ that is produced by the reaction of glucose with oxygen and water in the presence of GOx may interfere with the transfer of electrons to the electrode, possibly affecting the accuracy of the measured current and glucose concentration. And, because it is still operating at a positive potential in anodic (oxidation) mode, there may still be some interference from other oxidizable species that may be present in the sample

In the presence of oxygen and small concentrations of glucose, the enzyme such as GOx generates H₂O₂. In the presence of H₂O₂ and peroxidase (e.g. horseradish root peroxidase, HRP) a FcR is quickly oxidized to the corresponding ferricinium derivative (FcR⁺). When a cathodic (reduction) potential is applied to the working electrode, the FcR⁺ picks up electrons from the electrode (i.e. electrons are transferred from the electrode to the FcR⁺) and is reduced (i.e. reduced to the FcR form). However, many ferrocenes are also reduced by reaction with glucose in the presence of GOx, as shown in FIG. 4.

Thus, the reaction of the oxidized ferrocene with glucose in the presence of GOx to produce the reduced form of the ferrocene, interferes with the reduction of the oxidized ferrocene by transfer of electrons from the working electrode. The resulting current may therefore not accurately reflect the concentration of glucose in the sample. If the produced FcR⁺ is not involved in interfering reactions, then its measurement by electrochemical reduction determines the concentration of glucose in the solution. The interest of reversing the anodic electrochemical detection (oxidation) to the cathodic mode (reduction) is to escape the interference of many oxidizable products which are very often in the solutions of real samples to be tested.

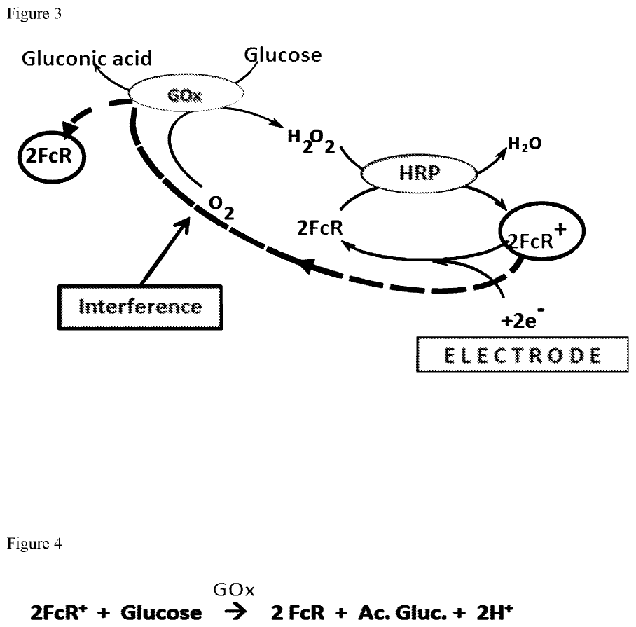

Kinetic studies on such a system have shown that competing reactions with different FcR can take place in the presence of glucose and GOx during the electrochemical detection (see FIG. 3). These reactions affect the electrochemical signal. Such interference is particularly detrimental when measuring samples with low concentrations of glucose (e.g. levels less than 0.02 mM).

To solve the interference problem of ferricinium derivatives (FcR⁺), it has been necessary to carry out kinetic studies of several ferricinium derivatives. Two methods were employed.

Cyclic voltammetry is a potentiodynamic method in which the potential at the working electrode is ramped linearly versus time, and after the set potential is reached, the potential at the working electrode is ramped in the opposite direction to return to the initial potential. The current at the working electrode is plotted versus the applied voltage. Chronoamperometry is the basic technique of many biosensors using electrochemical detection, and especially for the glucose measurement in anodic mode. Chronoamperometry is a potential step method in which the potential of the working electrode is stepped from a region in which nothing happens to a potential at which the redox reaction occurs, and held for a fixed period of time. The current is measured versus time. The potential step method is also the basis for the technique of pulse voltammetry. In pulse voltammetry, a combination of multiple steps of varying magnitude is applied and the current sampled in time to construct a current-voltage response.

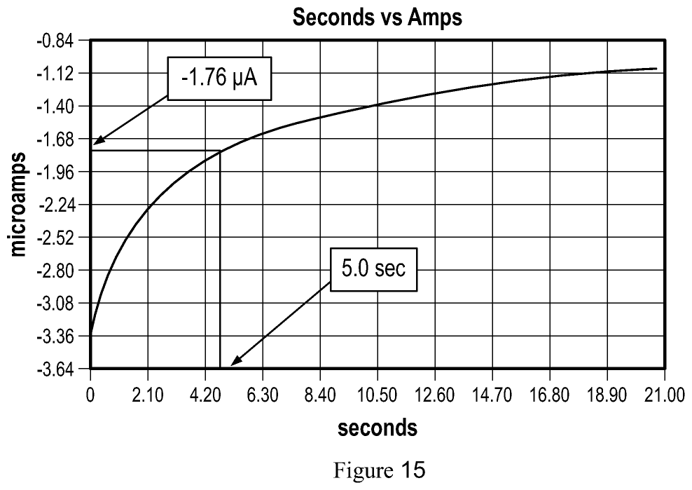

Preferably, chronoamperometry is used to measure the concentration of analytes in biological fluid samples. Chronoamperometry is an electrochemical technique in which the potential of the working electrode is fixed, and the resulting faradaic current (i) is monitored as a function of time (t^{1/2}) (See FIG. 15). The faradaic current decays as described in the Cottrell equation: i=k.i.t^{1/2}.

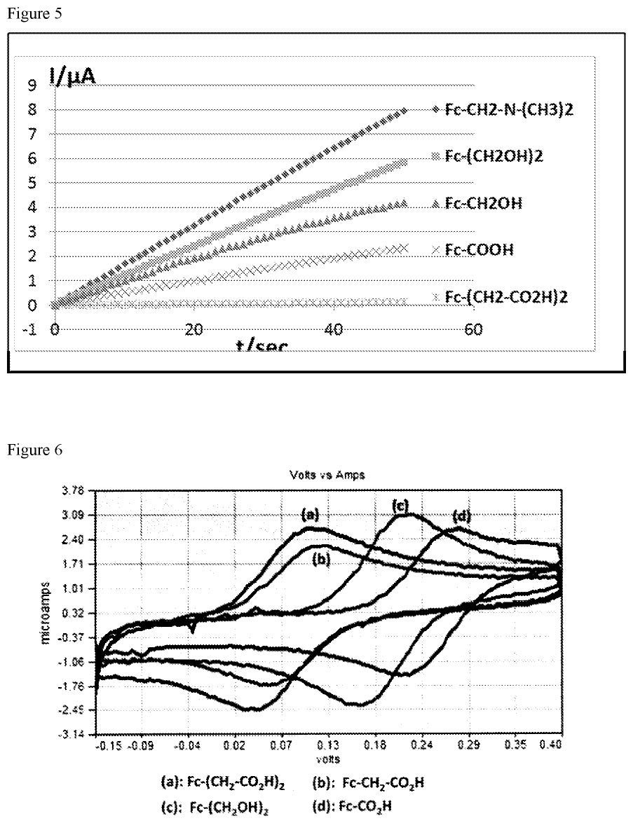

In one method, solutions of several FcR⁺ were prepared by preparative electro-synthesis oxidation of the corresponding FcR at +0.5 V on a platinum electrode vs. a saturated calomel electrode (SCE). Each of these solutions of FcR⁺ was contacted with a solution of glucose and GOx to see if they participated in the reaction as shown above. When glucose and GOx are added to a solution containing FcR⁺ derivative, the FcR⁺ that reacts with glucose in the presence of GOx is reduced (as shown in FIG. 4), and the analysis is initiated by oxidation using a rotating platinum electrode (at +0.5 V) to detect whether or not the FcR is generated. That is, the current generated by transfer of electrons from the reduced FcR to the platinum electrode is measured. The higher the oxidation current, the more interference may become an important factor, and the corresponding ferrocene may not be a good redox mediator for the biosensor. FIG. 5 shows the kinetics for five different ferrocene mediators, four of which generate, at different rates, the reaction which causes the interference, and which does not allow accurate cathodic measurements of glucose concentration. The only ferrocene mediator that does not react with glucose in the presence of GOx is [Fc-(CH₂—CO₂H)₂]⁺ derivative. Thus, [Fc-(CH₂—CO₂H)₂]⁺ could be an ideal mediator for the measurement of low concentrations of glucose in cathodic mode.

The second method used is based on studies of analytical electrochemistry, using cyclic voltammetry and chronoamperometry. The curves obtained with these two techniques allow, in a relatively short time, determination whether the ferrocene derivatives can function as good mediators in cathodic mode. FIG. 6 shows the superimposed cyclic voltammetric curves of four ferrocenes at a low concentration, using a very low speed scan (5 mV/sec) starting at −0.15 V to +0.4 V. The curves show that for the four products the reactions are reversible (FcR←-→FcR⁺+1 e⁻).

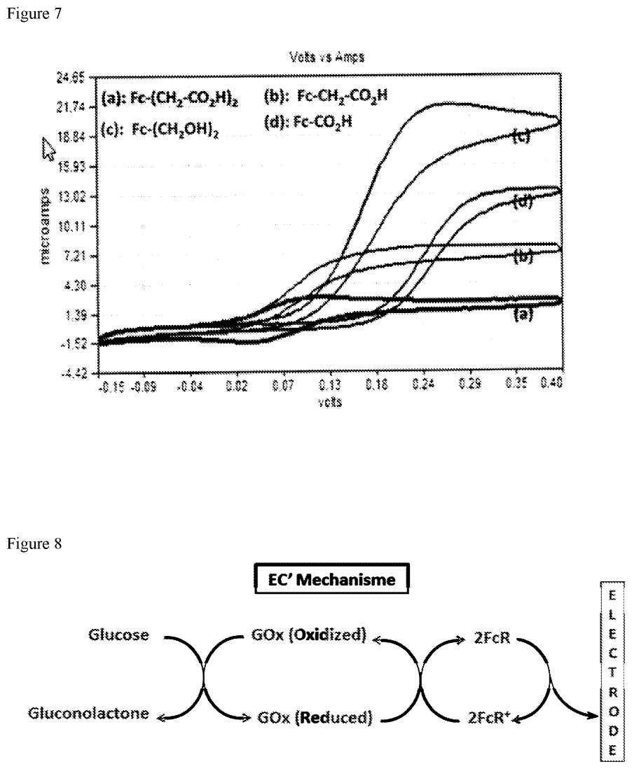

FIG. 7 shows the scanning at the same rate and with the same concentrations of ferrocenes as in FIG. 6, but in the presence of GOx and glucose. In this case, a single derivative, Fc-(CH₂—CO₂H)₂ is not affected by the presence of GOx and glucose. The other three ferrocenes behave differently: increase of the current of oxidation peak, and disappearance of the current of reduction peak. This type of electrochemical mechanism is called EC′ and is shown in FIG. 8. This mechanism is at the basis of the second generation of anodic biosensors for glucose measurement, and which interferes with the method of the present invention, using cathodic mode. Only the derivative Fc-(CH₂—CO₂H)₂ does not react according to the mechanism EC′, and therefore operates without any interference from glucose in the presence of GOx, which makes it a new and unique mediator for biosensors able to measure low concentrations of glucose (and other analytes) in cathodic mode.

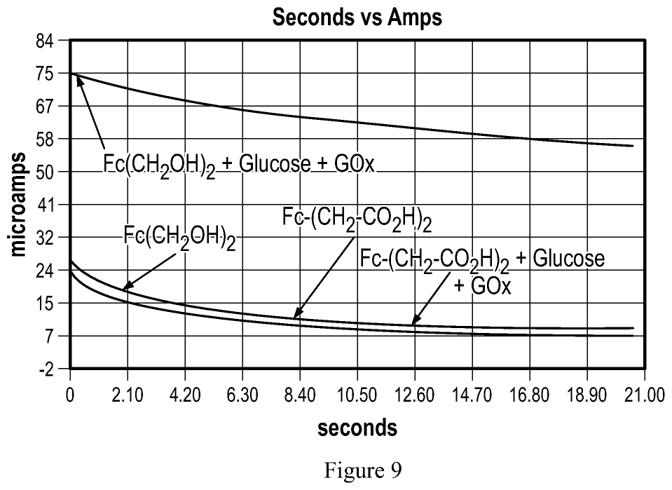

FIG. 9 shows the chronoamperometry curves recorded, at +0.3 V vs. Ag/AgCl reference electrode, under the same conditions as for the cyclic voltammetry in the absence, then in the presence of glucose oxidase, glucose, and respectively each of the two mediators Fc-(CH₂OH)₂ and Fc-(CH₂—CO₂H)₂. Again, it can be seen that Fc-(CH₂—CO₂H)₂ does not undergo the EC′ reaction, and is therefore an ideal redox mediator.

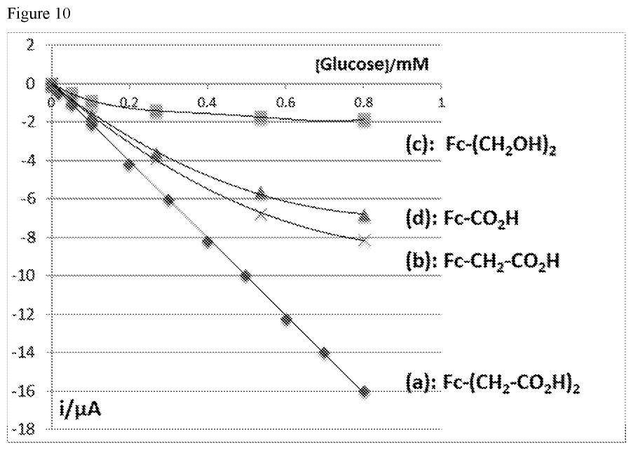

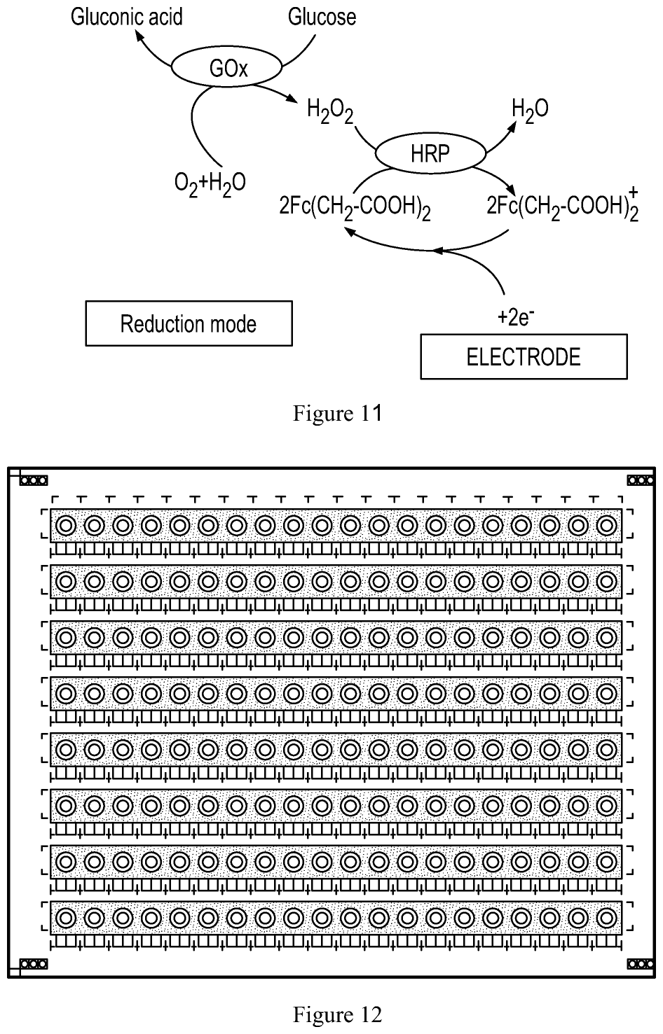

Whatever the method or technique used, Fc-(CH₂—CO₂H)₂ is currently a special mediator for measuring low glucose concentrations in cathodic mode. This makes it possible to avoid the oxidizable interfering products which particularly distort the measurements of the low glucose concentrations. FIG. 10 shows the glucose calibration curves using biosensors operating with different mediators. As can be seen in FIG. 10, the concentration response curve for Fc-(CH₂—CO₂H)₂ is linear, with a well-defined steep slope, and there is a clear difference in current depending on the concentration of glucose in the sample. FIG. 11 shows the reactions taking place on the biosensors using Fc-(CH₂—CO₂H)₂ as the mediator.

Based on these results, in a preferred embodiment, the electrochemical biosensors of the present invention comprise Fc-(CH₂—CO₂H)₂ as the redox mediator in a multi enzyme system (i.e. two or more enzymes). The electrochemical biosensors of the present invention work by chronoamperometry in reduction mode at negative potentials (less than 0 V vs. the Ag/AgCl reference electrode), and are capable of measuring low concentrations of glucose, for example, in the analysis of samples that are suspected to contain little or no glucose. Such biological fluid samples include saliva, sweat, tears, and other biological fluids.

Using chronoamperometry, the electrochemical biosensors of the present invention are capable of measuring very low concentrations of glucose, for example in a range of 0.005 mmol/L to 2.0 mmol/L. For example, the electrochemical biosensors of the present invention can measure the low levels of glucose typically present in saliva (0.02 mmol/L to 0.25 mmol/L), tears (0.05 mmol/L to 0.5 mmol/L), or sweat (0.277 mmol/L to 1.11 mmol/L).

For the majority of healthy individuals, normal blood sugar levels are between 4.0 mmol/L to 6.0 mmol/L (72 mg/dL to 108 mg/dL) when fasting, and up to 7.8 mmol/L (140 mg/dL) two hours after eating. For diabetics, blood sugar level targets are 4.0 mmol/L to 7.0 mmol/L for people with type 1 or type 2 diabetes before meals; under 9.0 mmol/L for people with type 1 diabetes after meals; and under 8.5 mmol/L for people with type 2 diabetes after meals.

Manufacturers of currently available monitors/biosensors for glucose measurement claim a range of 20 mg/dL to 600 mg/dL (1.1 mmol/L to 33.3 mmol/L). However, physiological range of glucose in saliva, tears, and sweat is much lower (see above). Therefore, currently available glucose monitors are not suitable for measuring the glucose levels in such biological fluids.

The electrochemical biosensors of the present invention are suitable for measuring a range of analytes in addition to glucose, such as, for example, sucrose.

In a preferred embodiment, the electrochemical cells of the present invention are prepared by screen-printing the electrodes onto a support substrate (see Examples 1 and 2). However, other print techniques, such as flexographic, gravure, lithographic, digital, etc. are also possible. Preferred support member substrates are plastic films (e.g. polybutylene terephthalate (PBT), polyethylene terephthalate (PET), polyvinyl chloride (PVC), polycarbonate, polyesters blended with polycarbonate, polypropylene, etc.), as well as paper and cellulosic substrates. The electrochemical cells of the present invention preferably comprise three electrodes: WE working electrode (carbon); CE counter electrode (carbon); and RE reference electrode (Ag/AgCl) (see Example 1). Different shapes, sizes, and materials can be used.

In addition to carbon, the working electrode and counter electrode may be made of any other suitable material. The material of which the working and reference electrodes are made must be conductive and chemically stable. Suitable materials include, but are not limited to carbon (e.g. graphite, graphene), platinum, gold, silicon compounds, and the like. The reference electrode must be made of a substance with a known and stable potential. Suitable materials for the reference electrode include, but are not limited to, silver/silver chloride (Ag/AgCl), and the like.

Different configurations may also be used. For example, the configuration could consist of a circular carbon working electrode surrounded by a carbon counter electrode and by an Ag/AgCl reference electrode (see Example 1). The configuration may also be only two electrodes. In a two electrode configuration, the working electrode could be surrounded by or face to face with, the reference electrode which also serves as the counter electrode (see Example 2). Multiple biosensors (transducers) are printed on a substrate card. The biosensors are die-cut after deposition of the bio-cocktails.

The surface of each glucose electrochemical biosensor is preferably modified by depositing a bio-cocktail containing the ingredients necessary for detecting and quantifying the analyte of interest. The bio-cocktail typically comprises one or more enzymes, and one or more redox mediators, in a buffered solution having a pH of about 6.5 to about 7.5. For example, the bio-cocktail for glucose may comprise 1,1′-ferrocene diacetic acid (i.e. Fc-(CH₂O₂H)₂), glucose oxidase (GOx), horseradish peroxidase (HRP), mutarotase (MUT) and a buffer solution, such as phosphate potassium salt (wherein the buffer solution has a pH of about 7). Other buffers can be used, such as, but not limited to, sodium acetate buffer, (N-morpholino)-ethane sulfonic acid, sodium buffer, citric acid buffer, and the like. At pH 7, glucose exists in solution in two cyclic hemiacetal forms (63.6% β-D-glucose and 36.4% α-D-glucose). Glucose oxidase reacts only with the β-D-glucose into D-glucono-1,5-lactone, which then hydrolyzes to gluconic acid. Mutarotase is an enzyme that converts α-D-glucose into β-D-glucose, which accelerates the total reaction and increase the signal of electrochemical current. For sucrose measurement, the same ingredients as for glucose are used, preferably with the addition of invertase (INV). Invertase is an enzyme that catalyzes the hydrolysis (breakdown) of sucrose into fructose and glucose. The generated glucose can then be measured with the glucose biosensor.

The bio-cocktail typically comprises GOx in an amount of about 100 UI/mL to about 2000 UI/mL. Preferably, the bio-cocktail comprises about 200 UI/mL to about 1000 UI/mL GOx. For example, the bio-cocktail may comprise GOx in an amount of about 100 UI/mL to about 1500 UI/mL; or about 100 UI/mL to about 1000 UI/mL; or about 100 UI/mL to about 500 UI/mL; or about 100 UI/mL to about 200 UI/mL; or about 200 UI/mL to about 2000 UI/mL; or about 200 UI/mL to about 1500 UI/mL; or about 200 UI/mL to about 1000 UI/mL; or about 200 UI/mL to about 500 UI/mL; or about 500 UI/mL to about 2000 UI/mL; or about 500 UI/mL to about 1500 UI/mL; or about 500 UI/mL to about 1000 UI/mL; or about 1000 UI/mL to about 2000 UI/mL; or about 1000 UI/mL to about 1500 UI/mL; or about 1500 UI/mL to about 2000 UI/mL.

The bio-cocktail typically comprises about 20 UI/mL to about 500 UI/mL HRP. Preferably, the bio-cocktail comprises about 50 UI/mL to about 300 UI/mL HRP. For example, the bio-cocktail may comprise HRP in an amount of about 20 UI/mL to about 400 UI/mL; or about 20 UI/mL to about 300 UI/mL; or about 20 UI/mL to about 200 UI/mL; or about 20 UI/mL to about 100 UI/mL; or about 20 UI/mL to about 50 UI/mL; or about 50 UI/mL to about 500 UI/mL; or about 50 UI/mL to about 400 UI/mL; or about 50 UI/mL to about 300 UI/mL; or about 50 UI/mL to about 200 UI/mL; or about 50 UI/mL to about 100 UI/mL; or about 100 UI/mL to about 500 UI/mL; or about 100 UI/mL to about 400 UI/mL; or about 100 UI/mL to about 300 UI/mL; or about 100 UI/mL to about 200 UI/mL; or about 200 UI/mL to about 500 UI/mL; or about 200 UI/mL to about 400 UI/mL; or about 200 UI/mL to about 300 UI/mL; or about 300 UI/mL to about 500 UI/mL; or about 300 UI/mL to about 400 UI/mL; or about 400 UI/mL to about 500 UI/mL.

The bio-cocktail typically comprises about 200 UI/mL to about 2000 UI/mL MUT. Preferably, the bio-cocktail comprises about 400 UI/mL to about 1600 UI/mL MUT. For example, the bio-cocktail may comprise MUT in an amount of about 100 UI/mL to about 1500 UI/mL; or about 100 UI/mL to about 1000 UI/mL; or about 100 UI/mL to about 500 UI/mL; or about 100 UI/mL to about 200 UI/mL; or about 200 UI/mL to about 2000 UI/mL; or about 200 UI/mL to about 1500 UI/mL; or about 200 UI/mL to about 1000 UI/mL; or about 200 UI/mL to about 500 UI/mL; or about 500 UI/mL to about 2000 UI/mL; or about 500 UI/mL to about 1500 UI/mL; or about 500 UI/mL to about 1000 UI/mL; or about 1000 UI/mL to about 2000 UI/mL; or about 1000 UI/mL to about 1500 UI/mL; or about 1500 UI/mL to about 2000 UI/mL.

The bio-cocktail typically comprises about 20 UI/mL to about 500 UI/mL INV. Preferably, the bio-cocktail comprises about 50 UI/mL to about 300 UI/mL INV. For example, the bio-cocktail may comprise INV in an amount of about 20 UI/mL to about 400 UI/mL; or about 20 UI/mL to about 300 UI/mL; or about 20 UI/mL to about 200 UI/mL; or about 20 UI/mL to about 100 UI/mL; or about 20 UI/mL to about 50 UI/mL; or about 50 UI/mL to about 500 UI/mL; or about 50 UI/mL to about 400 UI/mL; or about 50 UI/mL to about 300 UI/mL; or about 50 UI/mL to about 200 UI/mL; or about 50 UI/mL to about 100 UI/mL; or about 100 UI/mL to about 500 UI/mL; or about 100 UI/mL to about 400 UI/mL; or about 100 UI/mL to about 300 UI/mL; or about 100 UI/mL to about 200 UI/mL; or about 200 UI/mL to about 500 UI/mL; or about 200 UI/mL to about 400 UI/mL; or about 200 UI/mL to about 300 UI/mL; or about 300 UI/mL to about 500 UI/mL; or about 300 UI/mL to about 400 UI/mL; or about 400 UI/mL to about 500 UI/mL.

The bio-cocktail typically comprises about 0.01 mole/L to about 0.2 mole/L redox mediator. Preferably, the bio-cocktail comprises about 0.025 mole/L to about 0.1 mole/L redox mediator. For example, the bio-cocktail may comprise the redox mediator in an amount of about 0.01 mole/L to about 0.15 mole/L; or about 0.01 mole/L to about 0.10 mole/L; or about 0.01 mole/L to about 0.05 mole/L; or about 0.01 mole/L to about 0.025 mole/L; or about 0.025 mole/L to about 0.2 mole/L; or about 0.025 mole/L to about 0.15 mole/L; or about 0.025 mole/L to about 0.1 mole/L; or about 0.025 mole/L to about 0.05 mole/L; or about 0.05 mole/L to about 0.2 mole/L; or about 0.05 mole/L to about 0.15 mole/L; or about 0.05 mole/L to about 0.1 mole/L; or about 0.1 mole/L to about 0.2 mole/L; or about 0.1 mole/L to about 0.15 mole/L; or about 0.15 mole/L to about 0.2 mole/L.

The amounts of the ingredients typically depends on the activities of the enzymes used, the size of the transducer, the volume of the cocktail that will be deposited on the electrochemical biosensor, and also on the volume of the liquid (i.e. biological or experimental sample) containing the analyte to be measured, and on the measurement time.

The bio-cocktail is preferably deposited onto the working electrode by pipetting a small volume using a high precision pipette. Typically, the bio-cocktails are deposited in a volume of about 5 μL to about 50 μL. Preferably, the bio-cocktail is deposited in a volume of about 40 μL. For example, the bio-cocktail may be deposited in an amount of about 5 μL to about 45 μL; or about 5 μL to about 40 μL; or about 5 μL to about 35 μL; or about 5 μL to about 30 μL; or about 5 μL to about 25 μL; or about 5 μL to about 20 μL; or about 5 μL to about 15 μL; or about 5 μL to about 10 μL; or about 10 μL to about 50 μL; or about 10 μL to about 45 μL; or about 10 μL to about 40 μL; or about 10 μL to about 35 μL; or about 10 μL to about 30 μL; or about 10 μL to about 25 μL; or about 10 μL to about 20 μL; or about 10 μL to about 15 μL; or about 15 μL to about 50; or about 15 μL to about 45 μL; or about 15 μL to about 40 μL; or about 15 μL to about 35 μL; or about 15 μL to about 30 μL; or about μL to about 25 μL; or about 15 μL to about 20 μL; or about 20 μL to about 50 μL; or about 20 μL to about 45 μL; or about 20 μL to about 40 μL; or about 20 μL to about 35 μL; or about 20 μL to about 30 μL; or about 20 μL to about 25 μL; or about 25 μL to about 50 μL; or about 25 μL to about 45 μL; or about 25 μL to about 40 μL; or about 25 μL to about 35 μL; or about 25 μL to about 30 μL; or about 30 μL to about 50 μL; or about 30 μL to about 45 μL; or about 30 μL to about 40 μL; or about 30 μL to about 35 μL; or about 35 μL to about 50 μL; or about 35 μL to about 45 μL; or about 35 μL to about 40 μL; or about 40 μL to about 50 μL; or about 40 μL to about 45 μL; or about 45 μL to about 50 μL.

The pipette can be an automated pipetting machine. For example, the bio-cocktail can be pipetted onto the surface of the working electrode using an Innovadyne Nanodrop NS-2. The Innovadyne Nanodrop aspirates and dispenses a broad range of liquids, and features a software system that enables a wide range of applications and data manipulation. The Innovadyne Nanodrop offers high precision pipetting, with the advantages of non-contact dispensing, and a high dynamic range. The Innovadyne Nanodrop is very well adapted to quickly dispense accurate volumes of bio-cocktail solutions in the precise chosen place on the surface of the transducer. For the production of small number of biosensors it is possible to deposit the bio-cocktails using micropipettes.

After deposition, the bio-cocktails are dried on the transducer. Drying of the bio-cocktails on the biosensor is typically carried out in a freeze drier (used at room temperature). The transducers upon which the bio-cocktails are deposited are placed in the freeze drier with silica gel (dessicant) nearby. A low vacuum is created in the freeze drier by means of a pump and the biosensors are then left in the chamber under vacuum for about 12 to about 15 hours.

In certain embodiments, an insulator dielectric polymer is applied (e.g. via screen printing) over the printed electrodes. Typically, dielectric polymers are non-polar polymers. However, although less effective, polar polymers can also be used. Non-polar dielectric polymers include, but are not limited to, polyethylene (PE), polypropylene (PP), polystyrene (PS), and fluoropolymers, such as polytetraflouroethylene (PTFE), and the like. Polar dielectric polymers include, but are not limited to, poly(methyl methacrylate) (PMMA), polyvinyl chloride (PVC), polyamides (e.g. nylon), polycarbonate (PC), and the like.

The products dried on the transducers are protected by a medical grade, bio-compatible, open mesh fabric (e.g. from SEFAR Company). The open mesh fabrics are woven using monofilament yarns. The yarns are typically comprised of polyesters (e.g. polyethylene terephthalate (PET)) or polyamides (e.g. nylon). The sensors are cut using a die cutter (from Global Cutting Technologies Ltd., for example). Immediately after the sensors have been cut, they are placed in vials that protect them from humidity.

The disposable amperometric biosensors of the present invention can reliably measure very small amounts of glucose or sucrose in cathodic mode, which prevents the interference by many other oxidizable molecules present in biological samples. These biosensors are easy to use, have high sensitivity, and a broad linear range, with a shelf-life preferably of more than one year. The use of similar biosensors has been demonstrated for measuring glucose and sucrose in potato juices that contain very low amounts of sugars (e.g. less than 0.05 mmol/L, depending on the variety). The method of the present invention expands the fields of use to biological fluids (e.g. saliva, tears, and sweat). The use of these biosensors would be especially beneficial for humans and/or other animals that require monitoring of sugar levels for various reasons, for example diabetes.

For measuring analytes, such as glucose or sucrose, in saliva, it is recommended to rinse the mouth with water two or three times, wait approximately 1 minute, and then spit into a small clean container. A small volume of saliva (e.g. about 40 μL) is placed on the biosensor connected to the potentiostat. The chronoamperometric measurement (e.g. from 0 to 20 seconds) is conducted. The resulting current, by using a specific algorithm, provides the concentration of analyte.

For measuring analytes, such as glucose or sucrose, in tears, collect teardrops in a small, clean container. Pipette a small volume (e.g. about 40 μL), and place on the biosensor connected to the potentiostat. The chronoamperometric measurement (e.g. from 0 to 20 seconds) is conducted. The resulting current, by using a specific algorithm, provides the concentration of analyte.

In certain embodiments, the electrochemical biosensors and methods of the present invention are suitable to measure low concentrations of analytes, such as glucose and sucrose, in various food and agricultural products. Such food and agricultural products include, but are not limited to, potatoes, coffee, bread, etc. Recently, there has been concern about glucose and sucrose reacting in various food and agricultural products to produce acrylamides. Acrylamides are carcinogenic. Therefore, it would be advantageous to test different foods to determine which ones have concentrations of glucose and sucrose that could lead to the production of acrylamides, especially when the foods are heated at high temperatures.

### EXAMPLES

The following examples illustrate specific aspects of the present invention, and are not intended to limit the scope thereof in any respect and should not be so construed. The following examples illustrate preferred embodiments, but one of ordinary skill in the art will understand that other embodiments following with the scope and spirit of the invention may also be used.

### Method of Printing Electrodes

The support member substrate was prepared from polybutylene terephthalate resin (Valox FR-1 from General Electric). The substrate was 500 μm thick, and cut into cards with dimensions of 300 mm by 235 mm. Before use, the substrate was cured at 110° C. for 1 hour. A full card of Valox substrate has 160 screen printed transducers (see FIG. 12).

The electrodes were screen printed in separate layers, with a first printed layer, a second printed layer, and in some embodiments a third printed layer. Screen printing was carried out on a DEK HORIZON printer. After each printing step, the paste was dried in a box oven. Drying conditions for Ag/AgCl and carbon/graphite pastes were 60° C. for 30 minutes. The pastes could also be dried at 130° C. for 10 minutes. The drying conditions for the dielectric polymer were 80° C. for 30 minutes. The dielectric polymer could also be dried at 130° C. for 10 minutes.

In these examples, the dimensions of the transducers were 13.2 mm by 27.6 mm, with a circular working electrode of 6 mm in diameter. It is to be understood that other dimensions could be used, depending on the conditions of use for which the transducers are intended.

### Bio-Cocktails

Bio-cocktails are the solutions that contain the mixture of ingredients that allow selective detection, and quantification of, the analyte to be measured. A determined amount of the bio-cocktail was deposited on the surface of the transducer, followed by a low temperature drying step.

### Bio-Cocktail for Glucose Biosensor

The bio-cocktail for glucose measurement was a phosphate buffered solution, having a pH of 7.0 to 7.5. The enzymes and redox mediator were mixed into the phosphate buffered solution. The cocktail comprised 800 UI/mL of the enzyme glucose oxidase (GOx); 1600 UI/mL of the enzyme mutarotase (MUT); 200 UI/mL of the enzyme horseradish peroxidase (HRP); and 0.025 mole/L of the redox mediator 1,1′-ferrocene diacetic acid (i.e. Fc-(CH₂O₂H)₂).

### Bio-Cocktail for Sucrose Biosensor

The bio-cocktail for sucrose measurement was the same as that of the glucose bio-cocktail, with the addition of 200 UI/mL invertase (INV).

### Deposition of Bio-Cocktails

The bio-cocktails were deposited on the working electrodes of the electrochemical cells (on the screen printed transducers). The bio-cocktails were applied in a volume of 5 μL, using an Innovadyne Nanodrop NS-2, which aspirates and dispenses a broad range of liquids, and features a software system that enables a wide range of applications and data manipulation.

### Example 1. Electrochemical Cell (Transducer) Made of Three Electrodes

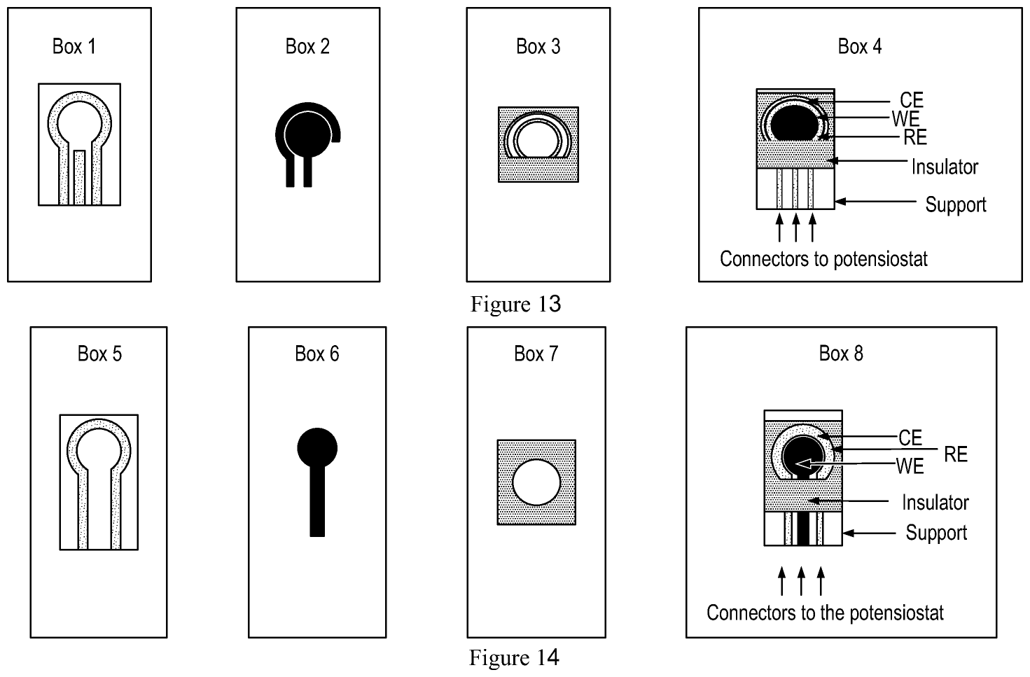

An electrochemical cell comprising three electrodes was produced. The electrodes were a working electrode (WE) made of carbon, and counter electrode (CE) made of carbon, and a reference electrode (RE) made of Ag/AgCl. The support substrate was Valox-FR1 from General Electric. FIG. 13 shows the printed layers of the Example 1 electrochemical cell.

A first screen printed layer of Ag/AgCl paste was applied to the substrate (FIG. 13, Box 1). The Ag/AgCl layer acts as the reference electrode, and ensures the electrical contacts of the three electrodes with the connectors to communicate with the potentiostat.

A second screen printed layer of carbon/graphite paste forms the circular working electrode, and the surrounding counter electrode (FIG. 13, Box 2).

A third screen printed layer of insulator dielectric polymer was applied to limit the geometry of the electrochemical cell, and to isolate the electrodes from one another (FIG. 13, Box 3).

The final electrochemical cell configuration is shown in FIG. 13, Box 4.

### Example 2. Electrochemical Cell (Transducer) Made of Two Electrodes

An electrochemical cell comprising two electrodes was prepared. The electrodes were a carbon WE and a dual counter and reference electrode (CE/RE) made of Ag/AgCl. FIG. 14 shows the electrochemical cell of Example 2.

A first screen printed layer (FIG. 14, Box 5) of Ag/AgCl paste on the substrate (Valox-FR1 from General Electric) acts simultaneously as a reference electrode and a counter electrode, and ensures the electrical contact of both electrodes with the potentiostat.

A second screen printed layer (FIG. 14, Box 6) of carbon/graphite forms the WE and its connector to the potentiostat.

A third screen printed layer (FIG. 14, Box 7) of insulator dielectric polymer limits the geometry of the electrochemical cell, and isolates the electrodes from one another.

The final electrochemical cell is shown in FIG. 14, Box 8.

### Example 3. Measurement of Glucose Concentration in Samples

A test for saliva analysis in the present study was performed using the biosensor described in this patent. This test was performed in the morning by a subject who was fasting.

The subject rinsed his mouth twice with water and after a short time without swallowing, he expectorated his saliva in a small clean container. 40 μL of saliva were deposited on the surface of the biosensor connected to a potentiostat whose potential was fixed at 0 V vs the reference Ag/AgCl. The chronoamperometry was then started and the current curve was recorded as a function of time in seconds (see FIG. 15). The current measured at 5 seconds allows the glucose concentration to be determined using the calibration curve (e.g. see linear curve (a) in FIG. 10).

The present invention has been described in detail, including the preferred embodiments thereof. However, it will be appreciated that those skilled in the art, upon consideration of the present disclosure, may make modifications and/or improvements on this invention that fall within the scope and spirit of the invention.

1. A method of measuring the concentration of one or more analytes in biological fluids, comprising:
a) placing a biological fluid sample on an electrochemical biosensor, wherein the electrochemical biosensor comprises:
i. a support member substrate;
ii. an electrochemical cell disposed on the support member substrate (transducer) comprising one or more working electrodes, a counter electrode, and a reference electrode;
iii. a bio-cocktail disposed on the electrochemical cell, wherein the bio-cocktail comprises:
a. one or more enzymatic catalysts selected from the group consisting of one or more oxidases (OX), a mutarotase (MUT), an invertase (INV), and one or more peroxidases (POX); and
b. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂;
b) applying a voltage to the electrochemical biosensor; and
c) measuring the current output;
wherein the current output is a function of the concentration of the analyte in the biological fluid.

2. The method of claim 1, wherein the oxidase is present in an amount of 4 UI or less per biosensor; and/or wherein the peroxidase is present in an amount of 1 UI or less per biosensor; and/or wherein the mutarotase is present in an amount of 16 UI or less per biosensor, and/or wherein the redox mediator is present in an amount of 0.037 mg or less per biosensor.

3. (canceled)

4. (canceled)

5. (canceled)

6. (canceled)

7. The method of claim 1, wherein at least one oxidase is glucose oxidase (GOx); and/or wherein at least one peroxidase is horseradish peroxidase (HRP); and/or wherein at least one enzyme catalyst is mutarotase; and/or wherein at least one enzyme catalyst is invertase.

8. (canceled)

9. (canceled)

10. The method of claim 7, wherein the analyte is glucose or sucrose.

11. (canceled)

12. (canceled)

13. The method of claim 1, wherein two analytes are measured.

14. The method of claim 13, wherein the analytes are glucose and sucrose.

15. The method of claim 1, wherein the bio-cocktail is disposed only on the working electrode.

16. The method of claim 1, wherein the voltage is applied using a potentiostat.

17. The method of claim 1, wherein the voltage is applied to one or more of the working electrodes.

18. The method of claim 1, wherein the support member substrate is selected from the group consisting of plastic films, paper, and cellulosic substrates.

19. The method of claim 18, wherein the support member substrate is a plastic film selected from the group consisting of polyethylene terephthalate, polyvinyl chloride, polycarbonate, polypropylene, and polyester.

20. The method of claim 1, wherein the biological fluid is saliva, sweat, tears, or other fluids secreted by mammals.

21. The method of claim 1, wherein the one or more working electrodes, counter electrode, and reference electrode are deposited onto the support member substrate by printing.

22. (canceled)

23. The method of claim 1, wherein one electrode is both the counter electrode and the reference electrode.

24. The method of claim 1, operable by chronoamperometry, cyclic voltammetry, or pulse voltammetry.

25. The method of claim 24, wherein the chronoamperometry is done in reduction mode.

26. The method of claim 1, wherein the reference electrode is an Ag/AgCl electrode.

27. The method of claim 1, wherein the working electrode and the counter electrode are carbon/graphite.

28. The method of claim 1, wherein concentrations of glucose and/or sucrose measured in the biological fluid are 0.005 to 1.1 mmol/L.

29. (canceled)

30. (canceled)

31. An electrochemical biosensor for measuring concentration of glucose in biological fluid samples, comprising:
a) a support member substrate;
b) an electrochemical cell disposed on the support member substrate (transducer) comprising one or more working electrodes, a counter electrode, and a reference electrode;
c) a bio-cocktail disposed on the electrochemical cell, wherein the bio-cocktail comprises:
i. glucose oxidase;
ii. mutarotase;
iii. peroxidase; and
iv. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂.

32. An electrochemical biosensor for measuring concentration of sucrose in biological fluid samples, comprising:
a) a support member substrate;
b) an electrochemical cell disposed on the support member substrate (transducer) comprising one or more working electrodes, a counter electrode, and a reference electrode;
c) a bio-cocktail disposed on the electrochemical cell, wherein the bio-cocktail comprises:
i. glucose oxidase;
ii. mutarotase;
iii. invertase;
iv. peroxidase; and
v. one or more ferrocene redox mediators selected from the group consisting of Fc-CH₂—N—(CH₃)₂, Fc-(CH₂OH)₂, Fc-CH₂OH, Fc-COOH, and Fc-(CH₂—CO₂H)₂.
